(19) United States
(12) Patent Application Publication
(10) Pub. No.: US 2023/0296630 A1
(43) Pub. Date: Sep. 21, 2023

(54) MULTIMARKER PANEL FOR THE ASSESSMENT OF SILENT BRAIN INFARCTS AND COGNITIVE DECLINE

(71) Applicant: Roche Diagnostics Operations, Inc., Indianapolis, IN (US)

(72) Inventors: Peter Kastner, Penzberg (DE); Vinzent Rolny, München (DE); Ursula-Henrike Wienhues-Thelen, Krailling (DE); Andre Ziegler, Läufelfingen (CH); David Conen, Ancaster (CA); Michael Kuehne, Binningen (CH); Stefan Osswald, Basel (CH)

(21) Appl. No.: 18/021,021

(22) PCT Filed: Aug. 12, 2021
(86) PCT No.: PCT/EP2021/072488

§ 371 Date: Feb. 13, 2023

(30) Foreign Application Priority Data
Aug. 14, 2020 (EP) 20191074.2

(51) Int. Cl. G01N 33/68

(52) U.S. Cl. CPC G01N 33/6896; G01N 2800/2871; G01N 2800/2814

(57) ABSTRACT

The present invention relates to a method for assessing whether a subject has experienced one or more silent infarcts in a subject, said method comprising a) determining the amounts of the biomarkers Osteopontin, cardiac Troponin, a natriuretic peptide and FABP-3 in a sample from the subject, b) comparing the amounts determined in step a) to references, and c) assessing whether a subject has experienced one or more silent infarcts. The present invention further relates to a method for predicting silent infarcts and/or cognitive decline, and methods for assessing and monitoring of the extent of silent small and large noncortical and cortical infarcts in a subject. Further encompassed by the present invention are the corresponding uses.

The present invention relates to a method for assessing whether a subject has experienced one or more silent infarcts, said method comprising a) determining the amounts of the biomarkers Osteopontin, cardiac Troponin, a natriuretic peptide and FABP-3 in a sample from the subject, b) comparing the amount determined in step c) to a reference, and assessing whether a subject has experienced one or more silent infarcts.

Also encompassed are methods for predicting silent infarcts and/or cognitive decline and for improving the prediction accuracy of a clinical risk score for silent brain infarcts and/or cognitive decline in a subject.

By combining the amounts of the biomarkers Osteopontin, cardiac Troponin, a natriuretic peptide and FABP-3 with the CHA₂D₂-VASc score, the prediction accuracy of a clinical risk score for silent brain infarcts and/or cognitive decline is improved. Furthermore, the present invention relates to a method for assessing the extent of silent large noncortical and cortical infarcts in a subject.

### BACKGROUND

Stroke ranks after ischemic heart disease second as a cause of lost disability—adjusted—life years in high-income countries and as a cause of dead worldwide. In order to reduce the risk of stroke, anticoagulation therapy appears the most appropriate therapy.

Atrial fibrillation (AF) is an important risk factor for stroke (Hart et al., Ann Intern Med 2007; 146(12): 857-67; Go AS et al. JAMA 2001; 285(18): 2370-5). Atrial fibrillation is characterized by irregular heart beating and often starts with brief periods of abnormal beating that can increase over time and may become a permanent condition. An estimated 2.7-6.1 million people in the United States have atrial fibrillation and approximately 33 million people globally (Chugh S. S. et al., Circulation 2014; 129:837-47). An early diagnosis of atrial fibrillation and an early prediction of the risk of stroke is highly desired because atrial fibrillation is an important risk factor for stroke and systemic embolism (Hart et al., Ann Intern Med 2007; 146(12): 857-67; Go AS et al. JAMA 2001; 285(18): 2370-5).

The diagnosis of heart arrhythmia such as atrial fibrillation typically involves determination of the cause of the arrhythmia, and the classification of the arrhythmia. Guidelines for the classification of atrial fibrillation according to the American College of Cardiology (ACC), the American Heart Association (AHA), and the European Society of Cardiology (ESC) are mainly based on simplicity and clinical relevance. The first category is called “first detected AF”. People in this category are initially diagnosed with AF and may or may not have had previous undetected episodes. If a first detected episode stops on its own in less than one week, but is followed by another episode later on, the category changes to “paroxysmal AF”. Although patients in this category have episodes lasting up to 7 days, in most cases of paroxysmal AF the episodes will stop in less than 24 hours. If the episode lasts for more than one week, it is classified as “persistent AF”. If such an episode cannot be stopped, i.e. by electrical or pharmacologic cardioversion, and continues for more than one year, the classification is changed to “permanent AF”.

Recent evidence suggests that patients with AF also face an increased risk of cognitive dysfunction/decline and dementia (Conen et al., J Am Coll Cardiol 2019; 73 989-99). Part of the association between AF and dementia is explained by the higher stroke risk among patients with AF. However the risk of dementia was also increased in patients with AF but without a clinical history of stroke. Clinically unrecognized cerebral infarcts (i.e. silent cerebral infarcts) or other brain lesions, such as white matter lesions might explain the association.

Silent large cortical and non-cortical infarcts (LNCCI) were associated with a cognitive dysfunction similar to that of overt strokes and corresponds to an approximate 10-year age difference in cognitive performance. Preventing silent brain infarcts seems therefore of major public health interest. Timely identification of these lesions is needed such that appropriate treatment measures can be initiated. However, brain magnetic resonance imaging (MRI) in all AF patients is unfeasible from a practical and financial perspective. Prediction tools are therefore needed to identify AF patients at high risk for silent brain lesions.

Silent large cortical and non-cortical infarcts (LNCCI) on magnetic resonance imaging are linked to several adverse outcomes, such as cognitive impairment and depression. For example, white matter changes have been reported to be associated with a decline in motor function in speed and fine motor coordination, and with many diseases including vascular dementia, dementia with Lewy bodies, and psychiatric disorders.

Biomarkers which allow for the assessment of stroke, silent brain infarcts and/or cognitive decline are highly required.

So far altered levels of Osteopontin, cardiac Troponin, a natriuretic peptide and FABP-3 have not yet been described for the assessment of stroke, for the assessment of silent infarcts or for the assessment of the extent of silent small and large noncortical or cortical infarcts (SNCI and LNCCI) in a subject.

Advantageously, it has been found in the studies underlying the present invention that Osteopontin, cardiac Troponin, a natriuretic peptide and FABP-3 are biomarker for the assessment of stroke and silent infarcts, and for the prediction of silent infarcts and/or cognitive decline. The determination of Osteopontin, cardiac Troponin, a natriuretic peptide and FABP-3 further allows for improving the prediction accuracy of a clinical risk score for silent brain infarcts.

Further, it was shown that the biomarkers Osteopontin, cardiac Troponin, a natriuretic peptide and FABP-3 positively correlates with the existence of silent small and large noncortical or cortical infarcts (SNCI and LNCCI) in patients.

Since the extent of SNCI and LNCCI can be caused by clinically silent strokes (Conen et al. 2019; Wang Y, Liu G, Hong D, Chen F, Ji X, Cao G. White matter injury in ischemic stroke. Prog Neurobiol. 2016;141:45-60), the biomarkers Osteopontin, cardiac Troponin, A natriuretic peptide and FABP-3 can be used for the assessment of the extent of SNCI, LNCCI and for the assessment whether a subject has experienced one or more silent strokes, i.e. clinically silent strokes, in the past.

### SUMMARY OF THE INVENTION

In a first aspect, the present invention relates to a method for assessing whether a subject has experienced one or more silent infarcts, said method comprising
a) determining the amounts of the biomarkers Osteopontin, cardiac Troponin, Aa natriuretic peptide and FABP-3 in a sample from the subject,
b) comparing the amounts determined in step a) to a reference, and
c) assessing whether a subject has experienced one or more silent infarcts.

In a second aspect, the present invention relates to a method for predicting silent infarcts and/or cognitive decline in a subject, said method comprising
a) determining the amounts of the biomarkers Osteopontin, cardiac Troponin, a natriuretic peptide and FABP-3 in a sample from the subject,
b) comparing the amount determined in step a) to a reference, and
c) predicting silent infarcts and/or cognitive decline in a subject,

In a third aspect, the present invention relates to a method for improving the prediction accuracy of a clinical risk score for silent brain infarcts and/ or cognitive decline for a subject, comprising the steps of
a) determining the amounts of the biomarkers Osteopontin, cardiac Troponin, a natriuretic peptide and FABP-3 in a sample from the subject, and
b) combining a value for the amounts of the biomarkers Osteopontin, cardiac Troponin, A natriuretic peptide and FABP-3 with the clinical risk score for silent brain infarcts, whereby the prediction accuracy of said clinical risk score for silent brain infarcts is improved.

In a fourth aspect, the present invention relates to a method for assessing the extent of silent small and large noncortical and cortical infarcts in a subject, said method comprising
a) determining the amounts of the biomarkers Osteopontin, cardiac Troponin, a natriuretic peptide and FABP-3 in a sample from the subject, and
b) assessing the extent of silent large noncortical or cortical infarcts in a subject based on the amount determined in step a).

In a fifth aspect, the present invention relates to a method for monitoring the extent of silent small and large noncortical or cortical infarcts and/or the cognitive function in a subject, comprising
a) determining the amounts of the biomarkers Osteopontin, cardiac Troponin, a natriuretic peptide and FABP-3 in a first sample from the subject,
b) determining the amounts of the biomarkers Osteopontin, cardiac Troponin, a natriuretic peptide and FABP-3 in a second sample from the subject which has been obtained after the first sample,
c) comparing the amounts of the biomarkers Osteopontin, cardiac Troponin, a natriuretic peptide and FABP-3 in the first sample to the amounts of the biomarkers Osteopontin, cardiac Troponin, a natriuretic peptide and FABP-3 in the second sample, and
d) monitoring the extent of silent small and large noncortical or cortical infarcts and/or the cognitive function and/or the cognitive function of the subject based on the results of step c).

In a seventh aspect, the present invention relates to method for a computer-implemented method for predicting stroke and/or silent infarct and/or cognitive decline in a subject, said method comprising
a) receiving at a processing unit a value for the amounts of the biomarkers Osteopontin, cardiac Troponin, A natriuretic peptide and FABP-3 in a sample from the subject,
b) processing the value received in step (a) with the processing unit, wherein said processing comprises retrieving from a memory one or more threshold values for the amounts of the biomarkers Osteopontin, cardiac Troponin, a natriuretic peptide and FABP-3 and comparing the value received in step (a) with the one or more threshold values, and
c) providing a prediction of silent infarct and/or cognitive decline via an output device, wherein said prediction is based on the results of step (b).

In an eight aspect, the present invention relates to the in-vitro use of the biomarkers Osteopontin, cardiac Troponin, a natriuretic peptide and FABP-3 or of agents, which binds to the amounts of the biomarkers Osteopontin, cardiac Troponin, a natriuretic peptide and FABP-3 for
a) predicting silent infarcts and/or cognitive decline in a subject,
b) assessing the extent of silent small and large noncortical or cortical infarcts t, or improving the prediction accuracy of a clinical stroke risk score for a subject.

### LIST OF FIGURES

FIG. 1 Receiver operating curves showing the accuracy of the models to predict the presence of large non-cortical and cortical infarcts. Biomarker include hs-Troponin, NT-proBNP, heart fatty-acid binding protein 3 and osteopontin. AUC=area under the curve; CI=confidence interval.

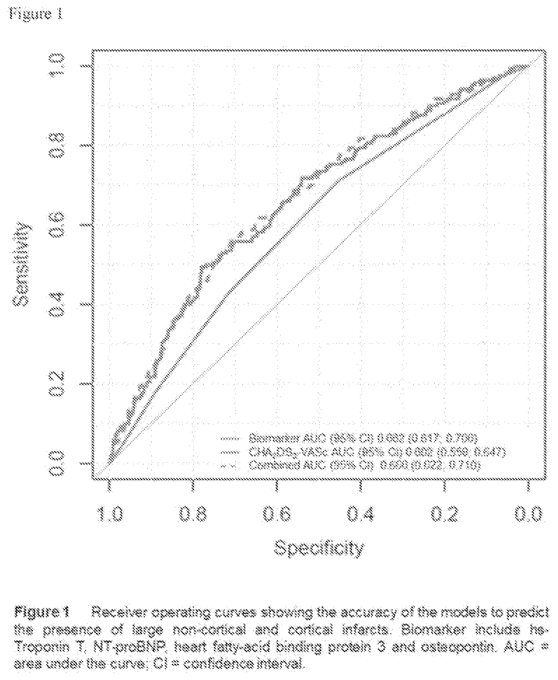

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

The method of the present invention, preferably, is an ex vivo or in vitro method. Moreover, it may comprise steps in addition to those explicitly mentioned above. For example, further steps may relate to sample pre-treatments or evaluation of the results obtained by the method. The method may be carried out manually or assisted by automation. Preferably, step (a), (b) and/or (c) may in total or in part be assisted by automation, e.g., by a suitable robotic and sensory equipment for the determination in step (a), or a computer-implemented comparison and/or prediction based on said comparison in step (b).

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Levels, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

As will be understood by those skilled in the art, the assessments as described herein, such as the assessment of stroke and/or silent infarcts, the assessment of the extent of silent large noncortical or small noncortical or cortical infarcts, the prediction of silent infarcts and/or cognitive decline, the improvement of the prediction accuracy of a clinical risk score for silent brain infarcts, the monitoring of the extent of silent large noncortical or cortical infarcts and/or the cognitive function, are usually not intended to be correct for 100% of the subjects.

In an embodiment, the prediction can be made for a statistically significant portion of subjects in a proper and correct manner. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%, or at least 99%. The p-values are, preferably, 0.1, 0.05, 0.01, 0.005, or 0.0001.

The term “Atrial Fibrillation” is well known in the art. As used herein, the term preferably refers to a supraventricular tachyarrhythmia characterized by uncoordinated atrial activation with consequent deterioration of atrial mechanical function. In particular, the term refers to an abnormal heart rhythm characterized by rapid and irregular beating. It involves the two upper chambers of the heart. In a normal heart rhythm, the impulse generated by the sino-atrial node spreads through the heart and causes contraction of the heart muscle and pumping of blood. In atrial fibrillation, the regular electrical impulses of the sino-atrial node are replaced by disorganized, rapid electrical impulses which result in irregular heart beats. Symptoms of atrial fibrillation are heart palpitations, fainting, shortness of breath, or chest pain. However, most episodes have no symptoms. On the electrocardiogram atrial fibrillation is characterized by the replacement of consistent P waves by rapid oscillations or fibrillatory waves that vary in amplitude, shape, and timing, associated with an irregular, frequently rapid ventricular response when atrioventricular conduction is intact.

The American College of Cardiology (ACC), American Heart Association (AHA), and the European Society of Cardiology (ESC) propose the following classification system (see Fuster (2006) Circulation 114 (7): e257-354 which herewith is incorporated by reference in its entirety, see e.g. FIG. 3 in the document): First detected AF, paroxysmal AF, persistent AF, and permanent AF.

All people with AF are initially in the category called first detected AF. However, the subject may or may not have had previous undetected episodes. A subject suffers from permanent AF, if the AF has persisted for more than one year. In particular, conversion back to sinus rhythm does not occur (or only with medical intervention). A subject suffers from persistent AF, if the AF lasts more than 7 days. The subject may require either pharmacologic or electrical intervention to terminate atrial fibrillation. Thus persistent AF occurs in episodes, but the arrhythmia does not typically convert back to sinus rhythm spontaneously (i.e. without medical invention). Paroxysmal atrial fibrillation, preferably, refers to an intermittent episode of atrial fibrillation which lasts not longer than 7 days and terminates spontaneously (i.e. without medical intervention). In most cases of paroxysmal AF, the episodes last less than 24 hours. Thus, whereas paroxysmal atrial fibrillation terminates spontaneously, persistent atrial fibrillation does not end spontaneously and requires electrical or pharmacological cardioversion for termination, or other procedures, such as ablation procedures (Fuster (2006) Circulation 114 (7): e257-354). The term “paroxysmal atrial fibrillation” is defined as episodes of AF that terminate spontaneously in less than 48 hours, more preferably in less than 24 hours, and, most preferably in less than 12 hours. Both persistent and paroxysmal AF may be recurrent.

As set forth above, the subject to be tested preferably suffers from paroxysmal, persistent or permanent atrial fibrillation.

Further, it is contemplated that the atrial fibrillation has been diagnosed previously in the subject. Accordingly, the atrial fibrillation shall be a diagnosed, i.e. a detected, atrial fibrillation.

Further, it is envisaged that the subject to be tested in accordance with the methods and use of the present invention, may have no known history of stroke and/or TIA (transient ischemic attack).

In an embodiment, the subject has no known history of stroke. In another embodiment, the subject has no known history of both stroke and TIA. Thus, the subject to be tested shall not have suffered from clinically recognized strokes and/or TIAs.

The term “assessing a silent infarct” as used herein, refers to the subject having silent stroke or received a silent infarct stroke. According to the present invention, the subject with silent stroke is at risk of developing a clinical stroke. The term “assessing a silent infarct” as used herein, further refers to a subject to diagnose silent infarcts, to determine the disease severity, to guide therapy (with objectives to therapy intensification/reduction), to predict disease outcome (risk prediction, e.g. stroke), therapy monitoring (e.g., effect of anti-coagulation drugs on levels of Osteopontin, cardiac Troponin, a natriuretic peptide and FABP-3) and therapy stratification of a subject (selection of therapy options; e.g. long-term from SWISS AF and selection).

The term “predicting the risk” as used herein, preferably, refers to assessing the probability according to which the subject will suffer from silent infarcts and/or cognitive decline.

Typically, it is predicted whether a subject is at risk (and thus at elevated risk) or not at risk (and thus at reduced risk) of suffering from silent infarcts and/or cognitive decline. Accordingly, the method of the present invention allows for differentiating between a subject at risk and a subject not at risk of suffering from silent infarcts and/or cognitive decline. Further, it is envisaged that the method of the present invention allows for differentiating between a subject who is a reduced, average, or elevated risk.

As set forth above, the risk (and probability) of suffering from silent infarcts and/or cognitive decline within a certain time window shall be predicted.

In an embodiment of the present invention, the prediction of silent infarcts and/or cognitive decline is determined after the sample to be tested has been obtained.

In another embodiment of the present invention, the predictive window, preferably, is an interval at least 1 month, at least 3 months, at least 6 months, at least 9 months, at least 1 year, at least 2 years, at least 3 years, at least 4 years, at least 5 years, at least 10 years, at least 15 years, or at least 20 years, or any intermitting time range.

In a preferred embodiment, the predictive window is a period of 1 month to 5 years. Thus, the risk to suffer from silent infarcts and/or cognitive decline within 1 month to 5 year is predicted. In a preferred embodiment, the predictive window a period of 1 month to 2 years. Preferably, the predictive window is a period of about one year. Most preferably, the predictive window might be a period of about two years. Thus, the risk of the subject to suffer from silent infarcts and/or cognitive decline within 2 years is predicted.

Preferably, said predictive window is calculated from the completion of the method of the present invention. More preferably, said predictive window is calculated from the time point at which the sample to be tested bas been obtained.

In a preferred embodiment, the expression “predicting the risk of silent infarcts and/or cognitive decline” means that the subject to be analyzed by the method of the present invention is allocated either into the group of subjects being at risk of suffering from silent infarcts and/or cognitive decline, or into the group of subjects not being at risk of suffering from silent infarcts and/or cognitive decline. Thus, it is predicted whether the subject is at risk or not at risk of suffering from silent infarcts and/or cognitive decline. As used herein “a subject who is at risk of suffering from silent infarcts and/or cognitive decline”, preferably has an elevated risk of suffering from silent infarcts and/or cognitive decline (preferably within the predictive window). Preferably, said risk is elevated as compared to the average risk in a cohort of subjects.

As used herein, “a subject who is not at risk of suffering from silent infarcts and/or cognitive decline”, preferably, has a reduced risk of suffering from silent infarcts and/or cognitive decline (preferably within the predictive window). Preferably, said risk is reduced as compared to the average risk in a cohort of subjects. A subject who is at risk of suffering from silent infarcts and/or cognitive decline preferably has a risk of suffering from silent infarcts and/or cognitive decline of at least 20% or more preferably of at least 30%, preferably, within a predictive window of about three years. A subject who is not at risk of suffering from silent infarcts and/or cognitive decline preferably has a risk of lower than 12%, more preferably of lower than 10% of suffering from said adverse event, preferably within a predictive window of two years.

The term “stroke” is well known in the art. The term, preferably, refers to ischemic stroke, in particular to cerebral ischemic stroke. A stroke which is predicted by the method of the present invention shall be caused by reduced blood flow to the brain or parts thereof which leads to an undersupply of oxygen to brain cells. In particular, the stroke leads to irreversible tissue damage due to brain cell death. Symptoms of stroke are well known in the art. Ischemic stroke may be caused by atherothrombosis or embolism of a major cerebral artery, by coagulation disorders or nonatheromatous vascular disease, or by cardiac ischemia which leads to a reduced overall blood flow. The ischemic stroke is preferably selected from the group consisting of atherothrombotic stroke, cardioembolic stroke and lacunar stroke. The term “stroke” does, preferably, not include hemorrhagic stroke.

Whether a subject suffers from stroke, in particular from ischemic stroke can be determined by well-known methods. Moreover, symptoms of stroke are well known in the art. E.g., stroke symptoms include sudden numbness or weakness of face, arm or leg, especially on one side of the body, sudden confusion, trouble speaking or understanding, sudden trouble seeing in one or both eyes, and sudden trouble walking, dizziness, loss of balance or coordination.

The term “Silent infarcts”: i.e. “silent cerebral infarcts” or “silent brain infarcts”, are known in the art and are, for example, described in Conen et al. (Conen et al., J Am Coll Cardiol 2019;73:989-99) which herewith is incorporated by reference with respect to its entire disclosure content. Silent infarcts are clinically silent infarcts in patients without a clinical history of stroke or transient ischemic attack. Accordingly, the subject to be tested shall have no known history of stroke and/or TIA (transient ischemic attack).

In a preferred embodiment, the risk of silent infarcts is predicted. The term, preferably, refers to a silent brain infarcts or an asymptomatic cerebral infarction (Krisai et al).

A silent infarct is a stroke that does not have any outward symptoms associated with stroke, and the patient is typically unaware they have suffered a stroke. Despite not causing identifiable symptoms, a silent stroke still causes damage to the brain and places the patient at increased risk for both transient ischemic attack and major stroke in the future. Silent infarcts are associated with subtle deficits in physical and cognitive function that commonly go unnoticed. A silent stroke often affects regions of the brain associated with various thought processes, mood regulation and cognitive functions and is a leading cause of cognitive decline or vascular cognitive impairment and may also lead to a loss of urinary bladder control. Silent infarcts typically cause lesions which are detected via the use of neuroimaging such as MRI.

The term “silent brain infarcts” is further defined as cerebral infarcts (LNCCIs and/or SNCIs) on brain MRI in patients without a history of stroke or TIA (Conen et. Al, 2019).

The term “LNCCI” is defined as large noncortical or cortical infarct, while the term “SNCI” is defined as small noncortical infarct.

In a preferred embodiment “subjects with large noncortical or cortical infarcts (LNCCI)” are assessed. The term “silent large noncortical or cortical infarcts (LNCCI)” are defined as hyperintense lesions on FLAIR>20 mm in diameter on axial sections and not involving the cortex. FLAIR=fluid-attenuated inversion recovery. These lesions are consistent with ischemic infarction in the territory of a perforating arteriole located in the white matter, internal or external capsule, deep brain nuclei, thalamus, or brainstem (Conen et al. 2019). Silent small and large noncortical or cortical infarcts (SNCI and LNCCI) on magnetic resonance imaging are linked to several adverse outcomes, such as cognitive impairment and depression. For example, white matter changes have been reported to be associated with a decline in motor function in speed and fine motor coordination, and with many diseases including vascular dementia, dementia with Lewy bodies, and psychiatric disorders.

The term “cognitive decline” as used herein is defined as a deterioration of memory, attention, and cognitive function. Alternatively to the term cognitive decline, the term cognitive dysfunction, the term cognitive impairment or the term dementia may be used.

The term preferably refers to a condition which can be characterized as a loss, usually progressive, of cognitive and intellectual functions, without impairment of perception or consciousness caused by a variety of disorders, but most commonly associated with structural brain disease. Cognitive testing may be done using the Montreal Cognitive Assessment (MoCA) as described in Conen et al. 2019. The term “cognitive function” relates to the assessment of cognitive function with scores as described in Conen et al. 2019 The Montreal Cognitive Assessment (MoCA) evaluates visuospatial and executive functions, confrontation naming, memory, attention, language, and abstraction. Patients can obtain a maximum of 30 points, with higher scores indicating better cognitive function. One point was added to the total test score if the patient had 12 years or less of formal education.

The most common type of dementia is Alzheimer's disease, which makes up 50% to 70% of cases. Other common types include vascular dementia (25%) dementia with Lewy bodies, and frontotemporal dementia. The term “dementia” includes, but is not restricted to AIDS dementia, Alzheimer dementia, presenile dementia, senile dementia, catatonic dementia, Lewy body dementia (diffuse Lewy body disease), multi-infarct dementia (vascular dementia), paralytic dementia, posttraumatic dementia, dementia praecox, vascular dementia.

In an embodiment, the term dementia refers to vascular dementia, Alzheimer's disease, dementia with Lewy bodies, and/or frontotemporal dementia. Thus, the risk to suffer from vascular dementia, Alzheimer's disease, dementia with Lewy bodies, and/or frontotemporal dementia is predicted.

In an embodiment, the risk to suffer from “Alzheimer's disease” is predicted. The term. “Alzheimer's disease” is well known in the art. Alzheimer's disease is a chronic neuro-degenerative disease that usually starts slowly and gradually worsens over time. As the disease advances, symptoms can include problems with language, disorientation, mood swings, loss of motivation, not managing self-care, and behavioural issues.

In an embodiment, the risk to suffer from “vascular dementia” is predicted. The term “vascular dementia” preferably refers to progressive loss of memory and other cognitive functions caused by vascular injury or disease within the brain. Thus, the term shall refer to the symptoms of dementia caused by problems of circulation of blood to the brain. It may occur after a silent brain infarct or after a stroke build up over time.

The methods of the present invention can be also used for the screening of larger populations of subjects. Therefore, it is envisaged, that at least 100 subjects, in particular at least 1000 subjects are assessed, e.g. with respect to the risk of silent infarcts. Thus, the amounts of the biomarkers Osteopontin, cardiac Troponin, A natriuretic peptide and FABP-3 are determined in samples from at least 100, or in particular of from at least 1000 subjects. Moreover, it is envisaged that at least 10,000 subjects are assessed.

The term “Anticoagulation therapy” is preferably a therapy which aims to reduce the risk of anticoagulation in said subject. Administration of at least one anticoagulant shall aim to reduce or prevent coagulation of blood and related stroke. In a preferred embodiment, at least one anticoagulant is selected from the group consisting of heparin, a coumarin derivative (i.e. a vitamin K antagonist), in particular warfarin or dicumarol, oral anticoagulants, in particular dabigatran, rivaroxaban or apixaban, tissue factor pathway inhibitor (TFPI), antithrombin III, factor IXa inhibitors, factor Xa inhibitors, inhibitors of factors Va and VIIIa and thrombin inhibitors (anti-IIa type).

In a particularly preferred embodiment, the anticoagulant is a vitamin K antagonist such as warfarin or dicumarol. Vitamin K antagonists, such as warfarin or dicumarol are less expensive, but need better patient compliance, because of the inconvenient, cumbersome and often unreliable treatment with fluctuating time in therapeutic range. NOAC (new oral anticoagulants) comprise direct factor Xa inhibitors (apixaban, rivaroxaban, darexaban, edoxaban), direct thrombin inhibitors (dabigatran) and PAR-1 antagonists (vorapaxar, atopaxar),

If the test subject is on anticoagulation therapy, and if the subject has been identified not to be at risk to suffer from silent infarcts (by the method of the present invention) the dosage of anticoagulation therapy may be reduced. Accordingly, a reduction of the dosage may be recommended. Be reducing the dosage, the risk to suffer from side effects (such as bleeding) may be reduced.

The term “Clinical stroke risk scores” are well known in the art. E.g. said scores are described in Kirchhof P. et al., (European Heart Journal 2016, 37: 2893-2962). In an embodiment, the score is CHA₂DS₂-VASc-Score. In another embodiment, the score is the CHADS₂ Score (Gage B F. Et al., JAMA, 285 (22) (2001), pp. 2864-2870) and ABC score, i.e. the ABC (age, biomarkers, clinical history) stroke risk score (Hijazi Z. et al., Lancet 2016; 387(10035): 2302-2311). All publications in this paragraph are herewith incorporated by reference with respect to their entire disclosure content.

Thus, in an embodiment, the clinical stroke risk score is the CHA₂DS₂-VASc-Score. In an alternative embodiment of the present invention, the clinical stroke risk score is the CHADS₂ Score.

The term “recommending” as used herein means establishing a proposal for a therapy which could be applied to the subject. However, it is to be understood that applying the actual therapy whatsoever is not comprised by the term. The therapy to be recommended depends on the outcome of, e.g. of the prediction by the method of the present invention.

The term “monitoring” as used herein, preferably, relates to assessing the disease progression as referred to herein elsewhere. Furthermore, the efficacy of a therapy for a patient may be monitored.

The “subject” to be tested in accordance with the methods and use of the present invention, preferably, is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the subject is a human subject. The terms “subject” and “patients” are used interchangeably herein.

In particular embodiments, the subject is a human patient. In embodiments, the patient is of any age. In embodiments, the patient is 50 years of age or older, in particular 60 years of age or older, and in particular 65 years of age or older. Further, is envisaged that the patient to be tested is 70 years of age or older.

In a preferred embodiment of the methods and uses of the present invention, the subject is 65 years of age or older. In another preferred embodiment, the subject is 70 years of age or older. In another embodiment, the subject is 75 years of age or older.

The term “sample” refers to a sample of a body fluid, to a sample of separated cells or to a sample from a tissue or an organ. Samples of body fluids can be obtained by well-known. techniques and include samples of blood, plasma, serum, urine, lymphatic fluid, sputum, ascites, saliva, lacrimal fluid, cerebrospinal fluid or any other bodily secretion or derivative thereof. Tissue or organ samples may be obtained from any tissue or organ by, e.g., biopsy. Separated cells may be obtained from the body fluids or the tissues or organs by separating techniques such as centrifugation or cell sorting. E.g., cell-, tissue- or organ samples may be obtained from those cells, tissues or organs which express or produce the biomarker. For example, the sample may be a myocardial tissue sample. Further, the sample may be a neural tissue sample, or a gut tissue sample. In some embodiments, the sample is a bone marrow sample. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

Thus, the sample may be a tissue sample. In a preferred embodiment, the tissue sample is a heart tissue sample, such as a myocardial tissue sample. In particular, the sample is a tissue sample from the right atrial appendage. In another preferred embodiment, the sample is a neural tissue sample, such as a brain tissue sample or spinal cord sample.

In another preferred embodiment, the sample is a blood (i.e. whole blood), serum or plasma sample. For example, the sample may be venous blood, serum or plasma sample. Alternatively, the sample may be a capillary blood sample (e.g. obtained from a finger). In some embodiments, the sample is peripheral blood sample. Serum is the liquid fraction of whole blood that is obtained after the blood is allowed to clot. For obtaining the serum, the clot is removed by centrifugation and the supernatant is collected. Plasma is the acellular fluid portion of blood. For obtaining a plasma sample, whole blood is collected in anticoagulant-treated tubes (e.g. citrate-treated or EDTA-treated tubes). Cells are removed from the sample by centrifugation and the supernatant (i.e. the plasma sample) is obtained.

Further, the sample may comprise stem cells, such as stem cells from the bone marrow or peripheral blood, lymphocytes, cardiomyocytes, neuronal cells or gut cells.

In some embodiments, the sample is a cerebrospinal fluid sample.

### Detection of a Biomarker

The biomarkers as referred to herein can be detected using methods generally known in the art. Methods of detection generally encompass methods to quantify the amount of a biomarker in the sample (quantitative method). It is generally known to the skilled artisan which of the following methods are suitable for qualitative and/or for quantitative detection of a biomarker. Samples can be conveniently assayed for, e.g., proteins using Westerns and immunoassays, like ELISAs, RIAs, fluorescence- and luminescence-based immunoassays and proximity extension assays, which are commercially available. Further suitable methods to detect biomarkers include measuring a physical of chemical property specific for the peptide or polypeptide such as its precise molecular mass or NMR spectrum. Said methods comprise, e.g., biosensors, optical devices coupled to immmoassays, biochips, analytical devices such as mass-spectrometers, NMR-analyzers, or chromatography devices. Further, methods include microplate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

For the detection of biomarker proteins as referred to herein a wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker.

Methods employing electrochemiluminescent labels are well-known. Such methods make use of the ability of special metal complexes to achieve, by means of oxidation, an excited state from which they decay to ground state, emitting electrochemiluminescence. For review see Richter, M. M., Chem. Rev. 2004:104: 3003-3036.

In an embodiment, the detection antibody (or an antigen-binding fragment thereof) to be used for measuring the amount of a biomarker is ruthenylated or iridinylated. Accordingly, the antibody (or an antigen-binding fragment thereof) shall comprise a ruthenium label. In an embodiment, said ruthenium label is a bipyridine-ruthenium(II) complex. Or the antibody (or an antigen-binding fragment thereof) shall comprise an iridium label. In an embodiment, said iridium label is a complex as disclosed in WO 2012/107419.

In an embodiment of the sandwich assay for the determination of Osteopontin, the assay comprises a biotinylated first monoclonal antibody that specifically binds Osteopontin (as capture antibody) and a ruthenylated F(ab′)2-fragment of a second monoclonal antibody that specifically binds Osteopontin as detection antibody). The two antibodies form sandwich immunoassay complexes with Osteopontin in the sample.

In a further embodiment of the sandwich assay for the determination of cardiac Troponin, the assay comprises a biotinylated first monoclonal antibody that specifically binds cardiac Troponin (as capture antibody) and a ruthenylated F(ab′)2-fragment of a second monoclonal antibody that specifically binds cardiac Troponin as detection antibody). The two antibodies form sandwich immunoassay complexes with cardiac Troponin in the sample.

In an embodiment of the sandwich assay for the determination of a vpeptide, the assay comprises a biotinylated first monoclonal antibody that specifically binds a natriuretic peptide (as capture antibody) and a ruthenylated F(ab′)2-fragment of a second monoclonal antibody that specifically binds a natriuretic peptide as detection antibody). The two antibodies form sandwich immunoassay complexes with a natriuretic peptide in the sample.

In an embodiment of the sandwich assay for the determination of FABP-3, the assay comprises a biotinylated first monoclonal antibody that specifically binds FABP-3 (as capture antibody) and a ruthenylated F(ab′)2-fragment of a second monoclonal antibody that specifically binds FABP-3 as detection antibody). The two antibodies form sandwich immunoassay complexes with FABP-3 in the sample.

Measuring the amount of a polypeptide may, preferably, comprise the steps of (a) contacting the polypeptide with an agent that specifically binds said polypeptide, (b) (optionally) removing non-bound agent, (c) measuring the amount of bound binding agent, i.e. the complex of the agent formed in step (a). According to a preferred embodiment, said steps of contacting, removing and measuring may be performed by an analyzer unit. According to some embodiments, said steps may be performed by a single analyzer unit of said system or by more than one analyzer unit in operable communication with each other. For example, according to a specific embodiment, said system disclosed herein may include a first analyzer unit for performing said steps of contacting and removing and a second analyzer unit, operably connected to said first analyzer unit by a transport unit (for example, a robotic arm), which performs said step of measuring.

The agent which specifically binds the biomarker (herein also referred to as “binding agent”) may be coupled covalently or non-covalently to a label allowing detection and measurement of the bound agent. Labeling may be done by direct or indirect methods. Direct labeling involves coupling of the label directly (covalently or non-covalently) to the binding agent. Indirect labeling involves binding (covalently or non-covalently) of a secondary binding agent to the first binding agent. The secondary binding agent should specifically bind to the first binding agent. Said secondary binding agent may be coupled with a suitable label and/or be the target (receptor) of a tertiary binding agent binding to the secondary binding agent. Suitable secondary and higher order binding agents may include antibodies, secondary antibodies, and the well-known streptavidin-biotin system (Vector Laboratories, Inc.). The binding agent or substrate may also be “tagged” with one or more tags as known in the art. Such tags may then be targets for higher order binding agents. Suitable tags include biotin, digoxygenin, His-Tag: Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium complexes, iridium complexes, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazoliun chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemoluminescence, which can be determined according to methods known in the art (e.g. using a light-sensitive film or a suit-able camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. A radioactive label can be detected by any method known and appropriate, e.g. a light-sensitive film or a phosphor imager.

The amount of a polypeptide may be, also preferably, determined as follows: (a) contacting a solid support comprising a binding agent for the polypeptide as described elsewhere herein with a sample comprising the peptide or polypeptide and (b) measuring the amount of peptide or polypeptide which is bound to the support. Materials for manufacturing supports are well-known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc.

In yet another aspect the sample is removed from the complex formed between the binding agent and the at least one marker prior to the measurement of the amount of formed complex. Accordingly, in an aspect, the binding agent may be immobilized on a solid support. In yet another aspect, the sample can be removed from the formed complex on the solid support by applying a washing solution.

“Sandwich assays” are among the most useful and commonly used assays encompassing a number of variations of the sandwich assay technique. Briefly, in a typical assay, an unlabeled (capture) binding agent is immobilized or can be immobilized on a solid substrate, and the sample to be tested is brought into contact with the capture binding agent. After a suitable period of incubation, for a period of time sufficient to allow formation of a binding agent-biomarker complex, a second (detection) binding agent labeled with a reporter molecule capable of producing a detectable signal is then added and incubated, allowing time sufficient for the formation of another complex of binding agent-biomarker-labeled binding agent. Any unreacted material may be washed away, and the presence of the biomarker is determined by observation of a signal produced by the reporter molecule bound to the detection binding agent. The results may either be qualitative, by simple observation of a visible signal, or may be quantitated by comparison with a control sample containing known amounts of biomarker.

The incubation steps of a typical sandwich assays can be varied as required and appropriate. Such variations include for example simultaneous incubations, in which two or more of binding agent and biomarker are co-incubated. For example, both, the sample to be analyzed and a labeled binding agent are added simultaneously to an immobilized capture binding agent. It is also possible to first incubate the sample to be analyzed and a labeled binding agent and to thereafter add an antibody bound to a solid phase or capable of binding to a solid phase.

The formed complex between a specific binding agent and the biomarker shall be proportional to the amount of the biomarker present in the sample. It will be understood that the specificity and/or sensitivity of the binding agent to be applied defines the degree of proportion of at least one marker comprised in the sample which is capable of being specifically bound. Further details on how the measurement can be carried out are also found elsewhere herein. The amount of formed complex shall be transformed into an amount of the biomarker reflecting the amount indeed present in the sample.

The terms “binding agent”, “specific binding agent”, “analyte-specific binding agent”, “detection agent”, “agent that binds to a biomarker” and “agent that specifically binds to a biomarker”, are used interchangeably herein. Preferably it relates to an agent that comprises a binding moiety which specifically binds the corresponding biomarker. Examples of “binding agents”, “detection agents”, “agents” are a nucleic acid probe, nucleic acid primer, DNA molecule, RNA molecule, aptamer, antibody, antibody fragment, peptide, peptide nucleic acid (PNA) or chemical compound. A preferred agent is an antibody which specifically binds to the biomarker to be determined. The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity (i.e. antigen-binding fragments thereof). Preferably, the antibody is a polyclonal antibody (or an antigen-binding fragment thereof). More preferably, the antibody is a monoclonal antibody (or an antigen-binding fragment thereof). Moreover, as described elsewhere herein, it is envisaged hat two monoclonal antibodies are used that bind at different positions of the biomarker polypeptide to be determined (in a sandwich immunoassay). Thus, at least one antibody is used for the determination of the amount of the biomarker.

The agent or detection agent shall specifically bind the biomarker Osteopontin, cardiac Troponin, a natriuretic peptide or FABP-3. The term “specific binding” or “specifically bind” refers to a binding reaction wherein binding pair molecules exhibit a binding to each other under conditions where they do not significantly bind to other molecules. The term “specific binding” or “specifically binds”, when referring to a protein or peptide as biomarker, preferably refers to a binding reaction wherein a binding agent binds to the corresponding biomarker with an affinity (“association constant” Kₐ) of at least 10⁷ M⁻¹. The term “specific binding” or “specifically binds” preferably refers to an affinity of at least 10⁸ M⁻¹ or even more preferred of at least 10⁹ M⁻¹ for its target molecule. The term “specific” or “specifically” is used to indicate that other molecules present in the sample do not significantly bind to the binding agent specific for the target molecule.

The term “amount” as used herein encompasses the absolute amount of a biomarker as referred to herein (such as Osteopontin, cardiac Troponin, a natriuretic peptide or FABP-3), the relative amount of concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts determined from biological read out systems in response to the peptides or intensity signals obtained from specifically bound ligands. It is to be understood that values correlating to the aforementioned amounts of parameters can also be obtained by all standard mathematical operations.

The term “comparing” as used herein refers to comparing the amount of the biomarkers (Osteopontin, cardiac Troponin, a natriuretic peptide or FABP-3) in the sample from the subject with the specific reference amount of the biomarker specified elsewhere in this description. It is to be understood that comparing as used herein usually refers to a comparison of corresponding parameters or values, e.g., an absolute amount is compared to an absolute reference amount while a concentration is compared to a reference concentration, or an intensity signal obtained from the biomarker in a sample is compared to the same type of intensity signal obtained from a reference sample. The comparison may be carried out manually or computer-assisted. Thus, the comparison may be carried out by a computing device. The value of the determined or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provides the desired prediction in a suitable output format.

In accordance with the present invention, the amounts of the biomarkers Osteopontin, cardiac Troponin, a natriuretic peptide and FABP-3 shall be compared to a reference, i.e. to a reference amount or to reference amounts. Accordingly, the reference is preferably a reference amount. The terms “reference amount” or “reference” are well understood by the: skilled person.

It is to be understood that the reference amount shall allow the prediction of silent infarcts and/or cognitive decline, in the improvement of the prediction accuracy of a clinical risk score for silent brain infarcts for a subject, in the assessment of the extent of silent large noncortical or cortical infarcts, in the assessment whether a subject has experienced one or more silent infarcts, in the monitoring of the extent of silent large noncortical or cortical infarcts and/or the cognitive function, and the diagnosis of atrial fibrillation in a subject as described herein elsewhere.

For example, in connection with the method for the prediction of the risk of silent infarcts and/or cognitive decline, the reference amount preferably refers to an amount which allows for allocation of a subject into either (i) the group of subjects who are at risk of suffering from silent infarcts and/or cognitive decline, or (ii) the group of subjects who are at risk of suffering from silent infarcts and/or cognitive decline. For example, in connection with the method for the diagnosis of silent infarcts the reference amount preferably refers to an amount which allows for allocation of a subject into either (i) the group of subjects suffering from silent infarcts or (ii) the group of subjects not suffering from silent infarcts. A suitable reference amount may be determined from a reference sample to be analyzed together, i.e. simultaneously or subsequently, with the test sample.

Reference amounts can, in principle, be calculated for a cohort of subjects as specified above based on the average or mean values for a given biomarker by applying standard methods of statistics. In particular, accuracy of a test such as a method aiming to diagnose an event, or not, is best described by its receiver-operating characteristics (ROC) (see especially Zweig M H, et al., Clin. Chem. 1993,39:561-577). The ROC graph is a plot of all the sensitivity versus specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus1−specificity for the complete range of thresholds suitable for making a distinction. On the y-axis is sensitivity, or the true-positive fraction, which is defined as the ratio of number of true-positive test results to the product of number of true-positive and number of false-negative test results. It is calculated solely from the affected subgroup. On the x-axis is the false positive fraction, or 1−specificity, which is defined as the ratio of number of false positive results to the product of member of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/1−specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper night corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis for a given event with a proper balance of sensitivity and specificity, respectively.

Accordingly, the reference to be used for the method of the present invention, i.e. a threshold which allows the respective assessment, such as the prediction of silent infarcts and/or cognitive decline, the prediction of silent infarcts and/or cognitive decline, the improvement of the prediction accuracy of a clinical risk score for silent brain infarcts for a subject, the assessment of the extent of silent large noncortical or cortical infarcts, the assessment whether a subject has experienced one or more silent infarcts, the monitoring of the extent of silent large noncortical or cortical infarcts and/or the cognitive function, can be generated, preferably, by establishing a ROC for said cohort as described above and deriving a threshold amount therefrom.

Dependent on a desired sensitivity and specificity for the assessment, the ROC plot allows deriving a suitable threshold. It will be understood that an optimal sensitivity is desired for e.g. excluding a subject being at risk of silent infarcts and/or cognitive decline (i.e. a rule out) whereas an optimal specificity is envisaged for a subject to be predicted to be at risk of silent infarcts and/or silent infarcts (i.e. a rule in)

The term “reference amount” herein refers to a predetermined value. Said predetermined value shall allow for the assessment as referred to herein, such as the prediction of silent infarcts and/or cognitive decline, the improvement of the prediction accuracy of a clinical risk score for silent brain infarcts for a subject, the assessment of the extent of silent large noncortical or cortical infarcts, the assessment whether a subject has experienced one or more silent infarcts, the monitoring of the extent of silent large noncortical or cortical infarcts and/or the cognitive function in a subject.

In the method for the prediction of the risk of silent infarcts and/or cognitive decline, for example, the reference, i.e. the reference amount shall allow for differentiating between a subject who is at risk of suffering from silent infarcts and/or cognitive decline and a subject who is not at risk of suffering from silent infarcts and/or cognitive decline.

The biomarkers as referred to herein are well known in the art.

Osteopontin (SPP1) is also known as BNSP, BSPI, OPN, early T-lymphocyte activation 1, nephropontin, bone sialoprotein I, early T-lymphocyte activation 1, urinary stone protein, uropontin is a secreted phosphoprotein I. Osteopontin plays a role in the attachment of osteoclasts to the mineralized bone matrix. Furthermore, Osteopontin also acts as a cytokine involved in enhancing production of interferon-gamma and interleukin-12 and reducing production of interleukin-10 and is essential in the pathway that leads to type I immunity. In animal models of ischemic brain infarcts high levels of OPN were observed [Chang et al. (2018) Liquefaction-of-the-Brain-following-Stroke-Shares-a-Similar-Molecular-and-Morphological-Profile-with-Atherosclerosis-and-Mediates-Secondary-Neurodegeneration-in-an-Osteopontin-Dependent-Mechanism, eNeuro 2018; 10.1523/ENEURO.0076-18.2018].

The Brain Natriuretic Peptid type peptide (herein also referred to as BNP-type peptide) is preferably selected from the group consisting of pre-proBNP, proBNP, NT-proBNP, and BNP. The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP). Preferably, brain natriuretic peptides according to the present invention are NT-proBNP, BNP, and variants thereof. BNP is the active hormone and has a shorter half-life than its respective inactive counterpart NT-proBNP. Preferably, the Brain Natriuretic Peptid-type peptide is BNP (Brain natriuretic peptide), and more preferably a natriuretic peptide (N-terminal of the prohormone brain natriuretic peptide).

The term “cardiac Troponin” refers to all Troponin isoforms expressed in cells of the heart and, preferably, the subendocardial cells. These isoforms are well characterized in the art as described, e.g. in Anderson 1995, Circulation Research, vol. 76, no 4: 681-686 and Ferrieres 1998, Clinical Chemistry, 44: 487-493. Preferably, cardiac Troponin refers to Troponin T and/or Troponin I, and, most preferably, to Troponin T. In a preferred embodiment, cardiac Troponin is hsTnT (high sensitive Troponin).

The term “FABP-3” as used herein refers to the fatty acid binding protein 3. FABP-3 is also known as heart fatty acid binding protein or heart type fatty acid binding protein (abbreviated H-FABP or hFABP-3). Preferably, the term also includes variants of FABP-3. FABP-3 as used herein, preferably, relates to human FABP-3. The DNA sequence of the polypeptide encoding the human FABP-3 polypeptide as well the protein sequence of human FABP-3 is well known in the art and was first described by Peeters et al. (Biochem. J. 276 (Pt 1), 203-207 (1991)). Moreover, the sequence of human H-FABP can be found, preferably, in Genbank entry U57623.1 (cDNA sequence) and AAB02555.1 (protein sequence). The major physiological function of FABP is thought to be the transport of free fatty acids, see e.g. Storch et al., Biochem. Biophys. Acta. 1486 (2000), 28-44. Other names for FABP-3 and H-FABP are: FABP-11 (fatty acid binding protein 11), M-FABP (muscle fatty acid-binding protein), MDGI (mammary-derived growth inhibitor), and O-FABP.

The term “determining” the amount of a biomarker as referred to herein (such as the biomarkers Osteopontin, cardiac Troponin, A natriuretic peptide and hFABP-3) refers to the quantification of the biomarker, e.g. to measuring the level of the biomarker in the sample, employing appropriate methods of detection described elsewhere herein. The terms “measuring” and “determining” are used herein interchangeably.

In an embodiment, the amount of a biomarker is determined by contacting the sample with an agent that specifically binds to the biomarker, thereby forming a complex between the agent and said biomarker, detecting the amount of complex formed, and thereby measuring the amount of said biomarker.

In embodiments, the level of biomarkers Osteopontin, cardiac Troponin, a natriuretic peptide and FABP-3 is determined using antibodies, in particular using monoclonal antibodies. In embodiments, step a) of determining the level of biomarkers Osteopontin, cardiac Troponin, a natriuretic peptide and FABP-3 in a sample of the patient comprises performing an immunoassay. In embodiments, the immunoassay is performed either in a direct or indirect format. In embodiments such immunoassays is selected from the group consisting of enzyme linked immunosorbent assay (ELISA), enzyme immunoassay (EIA), radioimmunoassay (RIA), or immuno assays based on detection of luminescence, fluorescence, chemiluminescence or electrochemiluminescence.

In a particular embodiment, step a) of determining the level of Osteopontin in a sample of the subject comprises the steps of
i) incubating the sample of the subject with one or more antibodies specifically binding to Osteopontin, thereby generating a complex between the antibody and Osteopontin, and
ii) quantifying the complex formed in step i), thereby quantifying the level of Osteopontin in the sample of the subject.

In a further particular embodiment, step a) of determining the level of cardiac Troponin in a sample of the subject comprises the steps of
i) incubating the sample of the subject with one or more antibodies specifically binding to cardiac Troponin, thereby generating a complex between the antibody and cardiac Troponin, and
ii) quantifying the complex formed in step i), thereby quantifying the level of cardiac Troponin in the sample of the subject.

In a further particular embodiment, step a) of determining the level of a natriuretic peptide in a sample of the subject comprises the steps of
i) incubating the sample of the subject with one or more antibodies specifically binding to a natriuretic peptide, thereby generating a complex between the antibody and a natriuretic peptide, and
ii) quantifying the complex formed in step i), thereby quantifying the level of a natriuretic peptide in the sample of the subject.

In a further particular embodiment, step a) of determining the level of FABP-3 in a sample of the subject comprises the steps of
i) incubating the sample of the subject with one or more antibodies specifically binding to FABP-3, thereby generating a complex between the antibody and FABP-3, and
ii) quantifying the complex formed in step i), thereby quantifying the level of FABP-3 in the sample of the subject.

In particular embodiments, in step i) the sample is incubated with two antibodies, specifically binding to the biomarker to be determined. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first anti-antibody/biomarker/second anti-biomarker antibody complex. As the skilled artisan will readily appreciate it is nothing but routine experimentation to establish the time and conditions that are appropriate or that are sufficient for the formation of a complex either between the specific anti-biomarker antibody and the biomarker antigen/analyte (=anti-biomarker complex) or the formation of the secondary, or sandwich complex comprising the first antibody to the biomarker, biomarker (the analyte) and the second anti-biomarker antibody (=anti-biomarker antibody/biomarker/second anti-biomarker antibody complex)

The detection of the anti-biomarker antibody/biomarker complex can be performed by any appropriate means. The detection of the first anti-biomarker antibody/biomarker/second anti-biomarker antibody complex can be performed by any appropriate means. The person skilled in the art is absolutely familiar with such means/methods.

In certain embodiments a sandwich will be formed comprising a first antibody to the biomarker, biomarker (analyte) and the second antibody to the biomarker, wherein the second antibody is detectably labeled.

In one embodiment a sandwich will be formed comprising a first antibody to the biomarker, the biomarker (analyte) and the second antibody to biomarker, wherein the second antibody is detectably labeled and wherein the first anti-biomarker antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

In a particular embodiment, an antigen in the sample, a biotinylated monoclonal biomarker-specific antibody and a monoclonal biomarker-specific antibody labeled with a ruthenium complex form a sandwich complex. After addition of streptavidin-coated microparticles, the complex becomes bound to the solid phase via interaction of biotin and streptavidin.

### DETAILED DESCRIPTION OF THE PRESENT INVENTION

The methods as referred to in accordance with the present invention includes methods which essentially consist of the aforementioned steps or methods which include further steps. Moreover, the method of the present invention, preferably, is an ex vivo and more preferably an in vitro method. Moreover, it may comprise steps in addition to those explicitly mentioned above. For example, further steps may relate to the determination of further markers and/or to sample pre-treatments or evaluation of the results obtained by the method. The method may be carried out manually or assisted by automation. Preferably, step (a), (b) and/or (c) may in total or in part be assisted by automation, e.g., by a suitable robotic and sensory equipment for the determination in step (a) or a computer-implemented calculation in step (b).

In a preferred embodiment of the methods and uses of the present invention, the subject to be tested suffers from atrial fibrillation. Atrial fibrillation may be paroxysmal, persistent or permanent atrial fibrillation. Thus, the subject may suffer from paroxysmal, persistent or permanent atrial fibrillation, In particular, it is envisaged that the subject suffers from paroxysmal, persistent or permanent atrial fibrillation. The best performance was observed in patients with persistent atrial fibrillation.

Thus, in an embodiment of the present invention, the subject suffers from paroxysmal atrial fibrillation. In another embodiment of the present invention, the subject suffers from persistent atrial fibrillation. In another embodiment of the present invention, the subject suffers from permanent atrial fibrillation.

In a first aspect, the present invention relates to a method for assessing stroke in a subject, comprising the steps of
a) determining the amounts of the biomarkers Osteopontin, cardiac Troponin, a natriuretic peptide and FABP-3 in a sample from the subject, and
b) comparing the amounts of the biomarkers Osteopontin, cardiac Troponin, a natriuretic peptide and FABP-3 to reference amounts, whereby stroke is to be assessed.

In a preferred embodiment, an amounts of biomarkers Osteopontin, cardiac Troponin, a natriuretic peptide and FABP-3 larger than the references is indicative for a subject who is suffering from stroke, wherein the amounts of biomarkers Osteopontin, cardiac Troponin, a natriuretic peptide and FABP-3 lower than the references is indicative for a subject who is not at suffering from stroke.

In a preferred embodiment, the amounts of biomarkers Osteopontin, cardiac Troponin, a natriuretic peptide and FABP-3 larger than the references is indicative for a subject who is suffering from stroke, wherein an amount of biomarkers Osteopontin, cardiac Troponin, a natriuretic peptide and FABP-3 lower than the references is indicative for a subject who is not at suffering from stroke.

In a further preferred embodiment, the subject may suffer from atrial fibrillation.

In a preferred embodiment, the subject is human. Furthermore, the sample of the subject is preferably a blood, serum, plasma or tissue sample.

In a second aspect, the present invention relates to a method for assessing whether a subject has experienced one or more silent infarcts, said method comprising
a) determining the amounts of the biomarkers Osteopontin, cardiac Troponin, a natriuretic peptide and FABP-3 in a sample from the subject,
b) comparing the amounts determined in step a) to references, and
c) assessing whether a subject has experienced one or more silent infarcts.

In a preferred embodiment, the amounts of biomarkers Osteopontin, cardiac Troponin, a natriuretic peptide and FABP-3 larger than the references is indicative for a subject who is suffering from one or more silent infarcts, wherein the amounts of biomarkers Osteopontin, cardiac Troponin, a natriuretic peptide and FABP-3 lower than the references is indicative for a subject who is not at suffering from one or more silent infarcts.

In a further preferred embodiment, the subject may suffer from atrial fibrillation.

In a preferred embodiment, the subject is human. Furthermore, the sample of the subject is preferably a blood, serum, plasma or tissue sample.

In a third aspect, the present invention relates to a method for predicting silent infarcts and/or cognitive decline in a subject, said method comprising
a) determining the amounts of the biomarkers Osteopontin, cardiac Troponin, a natriuretic peptide and FABP-3 in a sample from the subject,
b) comparing the amounts determined in step a) to references, and
c) predicting silent infarcts and/or cognitive decline in a subject.

In a preferred embodiment, the risk of silent infarcts is predicted. The term, preferably, refers to a silent brain infarcts or an asymptomatic cerebral infarction (Krisai et al.).

In a further preferred embodiment, a value for the amounts of the biomarkers Osteopontin, cardiac Troponin, a natriuretic peptide and FABP-3 is combined with the CHA₂D₂-VASc score, whereby the prediction accuracy of a clinical risk score for silent brain infarcts is improved.

In a preferred embodiment, cognitive decline is predicted. Alternatively, it may be predicted whether a subject is at risk of cognitive decline/dementia or not. The risk of a decline of cognitive function and dementia may be assessed by cognitive testing.

Preferably, an amounts of Osteopontin, cardiac Troponin, a natriuretic peptide and FABP-3 larger than the references are predictive for silent infarcts and/or cognitive decline in a subject, wherein the amounts of Osteopontin, cardiac Troponin, a natriuretic peptide and FABP-3 lower than the references are not predictive for silent infarcts and/or cognitive decline in a subject

In a preferred embodiment, a value for the amounts of the biomarkers Osteopontin, cardiac Troponin, a natriuretic peptide and FABP-3 is combined with the CHA₂D₂-VASc score, whereby the prediction accuracy of a clinical risk score for silent brain infarcts and/or cognitive decline is improved.

Furthermore, the risk of the subject to suffer from silent infarct and/or cognitive decline in a subject is predicted within 1 month to 5 years, such as within 1 year or within 2 years.

In a further preferred embodiment, the subject may suffer from atrial fibrillation.

Furthermore, the sample of the subject is preferably a blood, serum, plasma or tissue sample. In a preferred embodiment, the subject is human.

In a forth aspect, the present invention relates to a method for improving the prediction accuracy of a clinical stroke risk score for silent infarcts and/or cognitive decline for a subject, comprising the steps of
a) determining the amounts of the biomarkers Osteopontin, cardiac Troponin, a natriuretic peptide and FABP-3 in a sample from the subject, and
b) combining a value for the amounts of the biomarkers Osteopontin, cardiac Troponin, a natriuretic peptide and FABP-3 with the clinical stroke risk score, whereby the prediction accuracy of said clinical stroke risk score for silent infarcts and/or cognitive decline is improved.

In the studies underlying the present invention, it has been shown that the determination of Osteopontin, cardiac Troponin, a natriuretic peptide and FABP-3 allows for improving the prediction accuracy of a clinical stroke risk score for silent infarcts and/or cognitive decline for a subject. Thus, the combined determination of clinical stroke risk score for silent infarcts and/or cognitive decline and the amounts of the biomarkers Osteopontin, cardiac Troponin, a natriuretic peptide and FABP-3 allows for an even more reliable prediction of clinical stroke as compared to the determination of Osteopontin, cardiac Troponin, a natriuretic peptide and FABP-3 alone or the determination of the clinical stroke risk score alone. Moreover, risk scores recommended by ESC Guidelines are not sensitive enough and miss patients for anti-coagulation therapy. The present invention detects patients for anti-coagulation therapy with a higher probability than current stroke risk scores recommended by ESC Guidelines.

Accordingly, the method for predicting the risk of silent infarcts and/or cognitive decline may further comprise the combination of the amounts of the biomarkers Osteopontin, cardiac Troponin, a natriuretic peptide and FABP-3 with the clinical stroke risk score. Based on the combination of the amounts of the biomarkers Osteopontin, cardiac Troponin, a natriuretic peptide and FABP-3 with the clinical risk score, the risk of silent infarcts of the test subject is predicted.

Alternatively, the method may comprise obtaining or providing the value for the clinical stroke risk score. Preferably, the value is a number. In an embodiment, the clinical stroke risk score is generated by one of the clinically based tools available to physicians. Preferably the value provided by determining the value for the clinical stroke risk score for the subject. More preferably, the value for the subject is obtained from patient record data-bases and medical history of the subject. The value for the score therefore can be also determined using historical or published data of the subject.

In accordance with the present invention, the amounts of the biomarkers Osteopontin, cardiac Troponin, a natriuretic peptide and FABP-3 may be combined with the clinical stroke risk score for silent infarcts and/or cognitive decline. This means preferably that a value for the amounts of the biomarkers Osteopontin, cardiac Troponin, a natriuretic peptide and FABP-3 is combined with the clinical stroke risk score. Accordingly, the values are operatively combined to predict the risk of the subject to suffer from silent infarcts and/or cognitive decline. By combining the value, a single value may be calculated, which itself can be used for the prediction.

Clinical stroke risk scores are well known in the art. E.g. said scores are described in Kirchhof P. et al., (European Heart Journal 2016; 37: 2893-2962). In an embodiment, the score is CHA₂DS₂-VASc-Score. In another embodiment, the score is the CHADS₂ Score. (Gage BF. Et al., JAMA, 285 (22) (2001). pp. 2864-2870) and ABC score, i.e. the ABC (age, biomarkers, clinical history) stroke risk score (Hijazi Z. et al., Lancet 2016; 387(10035): 2302-2311) All publications in this paragraph are herewith incorporated by reference with respect to their entire disclosure content.

Thus, in an embodiment, the clinical stroke risk score is the CHA₂DS₂-VASc-Score. In an alternative embodiment of the present invention, the clinical stroke risk score is the CHADS₂ Score.

In a preferred embodiment, a value for the amounts of the biomarkers Osteopontin, cardiac Troponin, a natriuretic peptide and FABP-3 is combined with the CHA₂D₂-VASc score, whereby the prediction accuracy of a clinical risk score for silent brain infarcts and/or cognitive decline is improved.

In a further preferred embodiment, the above method for predicting the risk of silent infarcts and/or cognitive decline in a subject further comprises the step of recommending anticoagulation therapy or of recommending an intensification of anticoagulation therapy if the subject has been identified to be at risk to suffer from stroke (as described elsewhere herein).

The method may comprise the further step of c) improving prediction accuracy of said clinical stroke risk score based on the results of step b).

The definitions and explanations given herein above in connection with the method of the prediction of the risk of silent infarcts and/or cognitive decline preferably apply to the aforementioned method as well. E.g., it envisaged that the subject is a subject who has a known clinical stroke risk score for silent infarcts and/or cognitive decline. Alternatively, the method may comprise obtaining or providing the value for the clinical stroke risk score for silent infarcts and/or cognitive decline.

Furthermore, the risk of the subject to suffer from silent infarct and/or cognitive decline in a subject is predicted within 1 month to 5 years, such as within 1 year or within 2 years.

In an embodiment, the subject may suffer from atrial fibrillation. Furthermore, the sample of the subject is preferably a blood, serum, plasma or tissue sample. In a preferred embodiment, the subject is human.

In a fifth aspect of the present invention, the method relates to the assessing of the extent of silent small and large noncortical and cortical infarcts in a subject, said method comprising
a) determining the amounts of Osteopontin, cardiac Troponin, a natriuretic peptide and FABP-3 in a sample from the subject, and
b) assessing of the extent of silent large noncortical or cortical infarcts in a subject based on the amount determined in step a).

Interestingly, it was shown in the studies underlying the present invention that the biomarkers Osteopontin, cardiac Troponin, a natriuretic peptide and hFABP-3 can be used for estimating the risk, presence and/or severity of cerebrovascular injury as cause of cognitive decline and cognitive dysfunction in atrial fibrillation patients. Specifically, it was: shown that the biomarkers Osteopontin, cardiac Troponin, a natriuretic peptide and FABP-3 correlate with existence of silent large and small noncortical or cortical infarcts (LNCCI or SNCI) in patients. The term “LNCCI” is defined as large noncortical or cortical infarct, while the term “SNCI” is defined as small noncortical infarct.

In a preferred embodiment “subjects with silent large noncortical or cortical infarcts (LNCCI)” are assessed.

In an embodiment of the aforementioned methods, the subject may suffer from atrial fibrillation. Furthermore, the sample of the subject is preferably a blood, serum, plasma or tissue sample. In a preferred embodiment, the subject is human.

The higher the amounts of the biomarkers Osteopontin, cardiac Troponin, a natriuretic peptide and hFABP-3, the higher the extent of LNCCI or SNCI or WML (and vice versa). Therefore, the biomarkers Osteopontin, cardiac Troponin, a natriuretic peptide and FABP-3 can be used as a markers for assessing the extent of silent small and large noncortical or cortical infarcts and/or the cognitive function in a subject.

In a preferred embodiment, the subject to be tested suffers from atrial fibrillation. Furthermore, the risk of the subject to suffer from silent infarct and/or cognitive decline in a subject within 1 month to 5 years is predicted, such as within 1 year or within 2 years.

In a seventh aspect of the present invention, the method relates to the monitoring the extent of silent small and large noncortical or cortical infarcts and/or the cognitive function in a subject, comprising
a) determining the amounts of the biomarkers Osteopontin, cardiac Troponin, a natriuretic peptide and FABP-3 in a first sample from the subject,
b) determining the amounts of the biomarkers Osteopontin, cardiac Troponin, a natriuretic peptide and FABP-3 in a second sample from the subject which has been obtained after the first sample,
c) comparing the amounts of the biomarkers Osteopontin, cardiac Troponin, a natriuretic peptide and FABP-3 in the first sample to the amounts of the biomarkers Osteopontin, cardiac Troponin, a natriuretic peptide and FABP-3 in the second sample, and
d) monitoring the extent of silent small and large noncortical or cortical infarcts and/or the cognitive function and/or the cognitive function of the subject based on the results of step c).

In a embodiment of the present invention, the method further comprising the steps of
a) recommending anticoagulation therapy,
b) recommending an intensification of anticoagulation therapy,
c) intensified risk factor management and
d) care in specialized clinics.

The method of the present invention may aid personalized medicine. In a preferred embodiment, the method for predicting the risk of silent infarcts in a subject further comprises i) the step of recommending anticoagulation therapy, or ii) of recommending an intensification of anticoagulation therapy, if the subject has been identified to be at risk to suffer from silent infarcts. In another preferred embodiment, the method for predicting the risk of silent infarcts in a subject further comprises i) the step of initiating anticoagulation therapy, or ii) of intensifying anticoagulation therapy, if the subject bas been identified to be at risk to suffer from silent infarcts (by the method of the present invention).

In particular, the following applies:

If the subject to be tested does not receive anticoagulation therapy, the initiation of anticoagulation is recommended, if the subject has been identified to be at risk to suffer from silent infarcts. Thus, anticoagulation therapy shall be initiated.

If the subject to be tested already receives anticoagulation therapy, the intensification of anticoagulation is recommended, if the subject has been identified to be at risk to suffer from silent infarcts. Thus, anticoagulation therapy shall be intensified.

In a preferred embodiment, anticoagulation therapy is intensified by increasing the dosage of the anticoagulant, i.e. dosage of the currently administered coagulant.

In a particularly preferred embodiment, anticoagulation therapy is intensified by replacing the currently administered anticoagulant with a more effective anticoagulant. Thus, a replacement of the anticoagulant is recommended.

It has been described that better prevention in high risk patients is achieved with the oral anticoagulant apixaban versus the vitamin K antagonist warfarin as shown in Hijazi at al., The Lancet 2016 387, 2302-2311, (Figure 4).

Thus, it is envisaged that the subject to be tested is a subject who is treated with a vitamin K antagonist such as warfarin or dicumarol. If the subject has been identified to be at risk to suffer from silent infarcts (by the method of the present invention), the replacement of the vitamin K antagonist with an oral anticoagulant, in particular dabigatran, rivaroxaban or apixaban is recommended. Accordingly, the therapy with the vitamin K antagonist is discontinued and therapy with an oral anticoagulant is initiated.

The terms “subject” and “sample” have been defined above. The definitions apply accordingly. For example, it is envisaged that the subject suffers from atrial fibrillation. Further, the sample may be, for example, a blood, serum or plasma sample, or tissue sample.

The following preferably applies as diagnostic algorithm:

The amounts of Osteopontin, cardiac Troponin, a natriuretic peptide and FABP-3 larger than the references are indicative for a subject who has experienced one or more silent infarcts, and/or an amounts Osteopontin, cardiac Troponin, a natriuretic peptide and FABP-3 lower than the references are indicative for a subject who has not experienced silent infarcts.

The definitions given herein above, preferably, apply mutatis mutandis to the following:

The studies carried out in the studies of the present invention indicate that it would be possible to monitor a subject based on changes in the amounts of the biomarkers Osteopontin, cardiac Troponin, a natriuretic peptide and FABP-3. For example, the extent of silent small and large noncortical or cortical infarcts can be monitored, i.e. whether the extent of small and large noncortical infarcts or cortical infarcts increases, or not. Since an increase of the extent of silent small and large noncortical or cortical infarcts may be associated with a decrease of cognitive function, the determination of the biomarkers Osteopontin, cardiac Troponin, a natriuretic peptide and FABP-3 would also allow for monitoring the cognitive function of a subject.

Accordingly, the present further concerns a method for monitoring a subject, comprising
a) determining the amounts of the biomarkers Osteopontin, cardiac Troponin, a natriuretic peptide and FABP-3 in a first sample from the subject,
b) determining the amount of the biomarkers Osteopontin, cardiac Troponin, a natriuretic peptide and FABP-3 in a second sample from the subject which has been obtained after the first sample,
c) comparing the amounts of the biomarker Osteopontin, cardiac Troponin, a natriuretic peptide and FABP-3 in the first sample to the amount of the biomarker in the second sample, and
d) monitoring the subject based on the results of step c).

Also present invention further relates to the in vitro use of the biomarkers Osteopontin, cardiac Troponin, a natriuretic peptide and FABP-3 or of an agent which binds to the biomarkers Osteopontin, cardiac Troponin, a natriuretic peptide and FABP-3 for monitoring a subject. In some embodiments, the biomarkers Osteopontin, cardiac Troponin, a natriuretic peptide and FABP-3 or the agents are used in a first and a second sample from the subject.

The subject to be monitored may be a subject as defined in connection with the method for predicting the risk of silent infarcts and/or cognitive decline. For example, the subject may suffer from atrial fibrillation.

Preferably, the extent of silent small and/or large noncortical or cortical infarcts and/or the cognitive function of the subject is monitored. However, it is also envisaged to monitor morphological changes of the myocardial atrium, cerebral infarctions, cerebral microbleeds, progression of arrhythmia, progression of comorbidities (hypertension or diabetes) and/or progression of depressive symptoms. Alternatively, the amount of functional brain tissue may be monitored.

The monitoring shall be based on the comparison of the amounts of the biomarkers Osteopontin, cardiac Troponin, a natriuretic peptide and FABP-3 in a first sample to the amount of the biomarkers Osteopontin, cardiac Troponin, a natriuretic peptide and FABP-3 in the second sample. The “second sample” is understood as a sample which is obtained in order to reflect a change of the amounts of the biomarkers Osteopontin, cardiac Troponin, a natriuretic peptide and FABP-3 as compared to the amounts of the biomarkers Osteopontin, cardiac Troponin, a natriuretic peptide and FABP-3 in the first sample. Thus, second sample shall be obtained after the first sample. Preferably, the second sample is not obtained too early after the first sample (in order to observe a sufficiently significant change to allow for monitoring). In an embodiment, the second sample is obtained at least one month after the first sample. In another embodiment, the second sample is obtained one month after the first sample. In another embodiment, the second sample is obtained at least one or two years after the first sample. Further, it is envisaged that the second sample is obtained not more than 15 years, not more than 10 years, or, in particular, not more than five years after the first sample. Thus, the second sample may be obtained, e.g. at least one month, but not more than five years after the first sample.

Further, it is envisaged that the subject exhibited a silent infarct between the first and the second sample. The term “silent infarct” has been defined herein above.

Preferably, an increased amount, in particular a significantly increased amount of the biomarkers Osteopontin, cardiac Troponin, a natriuretic peptide and FABP-3 in the second sample as compared to first sample is indicative for an increase of the extent of silent infarcts LNCCI in the subject and/or for a decline of the cognitive function of the subject. Thus, the extent of LNCCI increased and/or the cognitive function declined between the first and the second sample. Significantly increased amounts of the biomarkers Osteopontin, cardiac Troponin, a natriuretic peptide and FABP-3 are to be understood an increase, which is larger than the average decrease in a group of control subjects. In some embodiments, an increase of the amounts of the biomarkers Osteopontin, cardiac Troponin, a natriuretic peptide and FABP-3 of at least 0.5% (e.g. per year) such as an increase of at least 1% (e.g. per year), is indicative for an increase of the extent of silent infarcts LNCCIs and/or for a decline of the cognitive function.

The definitions given herein above, preferably, apply mutatis mutandis to the following;

The present invention further relates to a method for diagnosing of the severity of cognitive decline in a subject who suffers from cognitive decline, said method comprising.
a) determining the amount of the biomarkers Osteopontin, cardiac Troponin, a natriuretic peptide and FABP-3 in a sample from the subject,
b) comparing the amount determined in step a) to references, and
c) diagnosing of the severity of cognitive decline in a subject, preferably, based on the results of step c).

The terms “subject” and “sample” have been defined above. The definitions apply accordingly. For example, it is envisaged that subject was in sinus rhythm at the time the sample has been obtained.

Diagnosis of cerebrovascular injury such as silent large noncortical or cortical infarcts and/or clinically silent infarcts (including size, location and types of lesions) is nowadays performed using magnetic resonance imaging (MRI) that is typically lengthy and costly. The determination of Osteopontin, cardiac Troponin, a natriuretic peptide and FABP-3, however would allow for a fast and cost-efficient pre-selection for cerebral MRI.

The methods of the present invention may further comprise the step of subjecting the patient who has been identified to be at risk of silent infarcts and/or cognitive decline, who has been identified to have a high extent of silent small and/or large noncortical or cortical infarcts, who has been identified to have experienced one or more silent infarcts in the past, and/or who has been diagnosed to suffer from AF, to Magnetic Resonance Imaging (MRI) of the brain, in particular to MRI for assessing cerebrovascular injury.

In an eighth aspect of the present invention, the method relates to the in vitro use of the biomarkers Osteopontin, cardiac Troponin, a natriuretic peptide and FABP-3 or of agents which binds to the biomarkers for the prediction of silent infarcts and/or cognitive decline in a subject.

The present invention further relates to the in vitro use of the biomarkers Osteopontin, cardiac Troponin, a natriuretic peptide and FABP-3 or of agents which binds to the biomarkers for the assessment of the extent of silent small and/or large noncortical or cortical infarcts in a subject.

The present invention further relates to the in vitro use of the biomarkers Osteopontin, cardiac Troponin, a natriuretic peptide and FABP-3 or of agents which binds to the biomarkers for the assessment whether a subject has experienced one or more silent infarcts.

The present invention further relates to the in vitro use of the biomarkers Osteopontin, cardiac Troponin, a natriuretic peptide and FABP-3 or of agents which binds to the biomarker for improving the prediction accuracy of a clinical stroke risk score for a subject.

Preferably, the aforementioned uses are in vitro uses. Thus, they are preferably carried out in a sample obtained from a subject. Moreover, the detection agent is, preferably, an antibody such as a monoclonal antibody (or an antigen binding fragment thereof) which specifically binds to the biomarker to be determined.

The methods of the present invention may be also carried out as computer-implemented methods.

In a ninth aspect of the present invention, the method relates to a computer-implemented method for the prediction of stroke and/or silent infarcts and/or cognitive decline in a subject, said method comprising
a) receiving at a processing unit a value for the amounts of the biomarkers Osteopontin, cardiac Troponin, a natriuretic peptide and FABP-3 in a sample from the subject,
b) processing the value received in step (a) with the processing unit, wherein said processing comprises retrieving from a memory one or more threshold values for the amounts of the biomarkers Osteopontin, cardiac Troponin, a natriuretic peptide and FABP-3 and comparing the values received in step (a) with the one or more threshold values, and
c) providing a prediction of silent infarcts and/or cognitive decline via an output device, wherein said prediction is based on the results of step (b).

The present invention further relates to a computer-implemented method for the assessment of the extent of silent large noncortical or cortical infarcts in a subject, said method comprising,
a) receiving at a processing unit a value for the amount of the biomarkers Osteopontin, cardiac Troponin, a natriuretic peptide and FABP-3 in a sample from the subject,
b) processing the value received in step (a) with the processing unit, wherein said processing comprises retrieving from a memory one or more threshold values for the amounts of the biomarkers Osteopontin, cardiac Troponin, a natriuretic peptide and FABP-3 and comparing the value received in step (a) with the one or more threshold values, and
c) providing an assessment of the extent of silent large noncortical or cortical infarcts in a subject via an output device, wherein said assessment is based on the results of step (b).

The present invention further relates to a computer-implemented method for the assessment whether a subject bas experienced one or more silent infarcts, said method comprising
a) receiving at a processing unit a value for the amounts of the biomarkers Osteopontin, cardiac Troponin, a natriuretic peptide and FABP-3 in a sample from the subject,
b) processing the value received in step (a) with the processing unit, wherein said processing comprises retrieving from a memory one or more threshold values for the amount of the biomarkers Osteopontin, cardiac Troponin, a natriuretic peptide and FABP-3 and comparing the value received in step (a) with the one or more threshold values, and
c) providing an assessment whether a subject has experienced one or more silent infarcts via an output device, wherein said assessment is based on the results of step (b).

In an embodiment of the methods of the present invention, information on the prediction, assessment, or diagnosis (according to the last step of the computer-implemented methods of the present invention) is provided via a display, configured for presenting the prediction, assessment, or diagnosis. For example, information may be provided whether the subject is at risk of silent infarcts and/or cognitive decline, or not. Further, recommendations for suitable therapeutic measures can be displayed.

In an embodiment of the methods of the present invention, the methods may comprise the further step of transferring the information on the assessment of the methods of the present invention to the subject's electronic medical records.

Alternatively, the assessment made in the last step of the methods of the present invention can be printed by a printer. The print-out shall contain information on whether the patient is at risk, or not at risk and/or a recommendation of a suitable therapeutic measure.

The present invention further relates to computer program including computer-executable instructions for performing the steps of the method according to the present invention, when the program is executed on a computer or computer network. Typically, the computer program specifically may contain computer-executable instructions for performing the steps of the method as disclosed herein. Specifically, the computer program may be stored on a computer-readable data carrier.

The present invention further relates to a computer program product with program code means stored on a machine-readable carrier, in order to perform the computer-implemented method according to present invention, such as the computer-implemented method for the prediction of stroke and/or cognitive decline, when the program is executed on a computer or computer network, such as one or more of the above-mentioned steps discussed in the context of the computer program. As used herein, a computer program product refers to the gram as a tradable product. The product may generally exist in an arbitrary format, such as in a paper format, or on a computer-readable data carrier. Specifically, the computer program product may be distributed over a data network.

The present invention further relates to a computer or computer network comprising at least one processing unit, wherein the processing unit is adapted to perform all steps of the computer-implemented method according to the present invention.

Yet, the present invention also contemplates:
A computer or computer network comprising at least one processing unit, wherein said processing unit is adapted to perform the method according to one of the embodiments described in this description,
a computer loadable data structure that is adapted to perform the method according to one of the embodiments described in this description while the data structure is being executed on a computer,
a computer script wherein the computer program is adapted to perform the method according to one of the embodiments described in this description while the program being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising glucose data measurements obtained from the individual as specified herein above, and
a data stream signal, typically encrypted, comprising an information providing an aid in the assessment of guidance obtained by the method of the invention.

In further embodiments, the present invention relates to the following items:

In the following, embodiments of the present invention are summarized. The definitions given herein above, preferably, apply to the following embodiments.

1. A method for assessing stroke in a subject, comprising the steps of
a) determining the amounts of the biomarkers Osteopontin, cardiac Troponin, a natriuretic peptide and FABP-3 in a sample from the subject, and
b) comparing the amounts of the biomarkers Osteopontin, cardiac Troponin, a natriuretic peptide and FABP-3 to a reference amount, whereby stroke is to be assessed.

2. A method for assessing whether a subject has experienced one or more silent infarcts, said method comprising
a) determining the amounts of the biomarkers Osteopontin, cardiac Troponin, a natriuretic peptide and FABP-3 in a sample from the subject,
b) comparing the amount determined in step a) to a reference, and
c) assessing whether a subject has experienced one or more silent infarcts.

3. The method of any one of claims 1 to 2, wherein the amounts of Osteopontin, cardiac Troponin, a natriuretic peptide and FABP-3 larger than the references is indicative for a subject who is suffering from stroke and/or silent infarct, and/or wherein the amounts of Osteopontin, cardiac Troponin, a natriuretic peptide and FABP-3 lower than the references is indicative for a subject who is not at suffering from stroke and/or silent infarct.

4. A method for predicting silent infarcts and/or cognitive decline in a subject, said method comprising
a) determining the amounts of the biomarkers Osteopontin, cardiac Troponin, a natriuretic peptide and FABP-3 in a sample from the subject,
b) comparing the amount determined in step a) to references, and
c) predicting silent infarcts and/or cognitive decline in a subject.

5. The method of any one of claims 2 to 4, wherein a value for the amounts of the biomarkers Osteopontin, cardiac Troponin, a natriuretic peptide and FABP-3 is combined with the CHA₂D₂-VASc score, whereby the prediction accuracy of a clinical risk score for silent brain infarcts and/or cognitive decline is improved.

6. A method for improving the prediction accuracy of a clinical risk score for silent infarcts and/or cognitive decline for a subject, comprising the steps of
a) determining the amounts of the biomarkers Osteopontin, cardiac Troponin, a natriuretic peptide and FABP-3 in a sample from the subject, and
b) combining a value for the amounts of the biomarkers Osteopontin, cardiac Troponin, a natriuretic peptide and FABP-3 with the clinical risk score for silent brain infarcts, whereby the prediction accuracy of said clinical risk score for silent brain infarcts is improved.

7. The method of claim 6, wherein the risk of the subject to suffer from silent infarct and/or cognitive decline in a subject within 1 month to 5 years is predicted, such as within 1 year or within 2 years.

8. A method for assessing of the extent of silent small and large noncortical and cortical infarcts in a subject, said comprising
a) determining the amounts of the biomarkers Osteopontin, cardiac Troponin, a natriuretic peptide and FABP-3 in a sample from the subject, and
b) assessing of the extent of silent large noncortical or cortical infarcts in a subject based on the amount determined in step a).

9. The method of any one of embodiments 1 or 8, wherein the subject suffers from atrial fibrillation.

10. The method of any one of embodiments 1 or 10, wherein the atrial fibrillation is paroxysmal or persistent atrial fibrillation.

11. The method of any one of embodiments 1 or 10, wherein the subject is human and/or wherein the sample is preferably blood, serum or plasma or wherein the sample is tissue sample.

12. The method of any one of embodiments 1 to 11, wherein the biomarkers Osteopontin, cardiac Troponin, a natriuretic peptide and FABP-3 is a polypeptide.

13. The method of any one of embodiments 1 to 12, wherein the subject is 65 years or older.

14. The method of any one of embodiments 1 to 13, wherein the subject has no known history of stroke and/or TIA (transient ischemic attack).

15. The method of embodiments 1 to 14, wherein the subject was in sinus rhythm at the time the sample has been obtained.

16. A method for monitoring the extent of silent small and large noncortical or cortical infarcts and/the cognitive function in a subject, comprising
a) determining the amounts of the biomarkers Osteopontin, cardiac Troponin, a natriuretic peptide and FABP-3 in a first sample from the subject,
b) determining the amounts of the biomarkers Osteopontin, cardiac Troponin, a natriuretic peptide and FABP-3 in a second sample from the subject which has been obtained after the first sample,
c) comparing the amounts of the biomarkers Osteopontin, cardiac Troponin, a natriuretic peptide and FABP-3 in the first sample to the amounts of the biomarkers Osteopontin, cardiac Troponin, a natriuretic peptide and FABP-3 in the second sample, and
d) monitoring the extent of silent small and large noncortical or cortical infarcts and/or the cognitive function and/or the cognitive function of the subject based on the results of step c).

17. The method of any one of claims 1 to 16, further comprising the steps of
e) recommending anticoagulation therapy,
f) recommending an intensification of anticoagulation therapy,
g) intensified risk factor management and
h) care in specialized clinics.

18. A computer-implemented method for predicting stroke and/or silent infarct and/or cognitive decline in a subject, said method comprising
a) receiving at a processing unit a value for the amounts of the biomarkers Osteopontin, cardiac Troponin, a natriuretic peptide and FABP-3 in a sample from the subject,
b) processing the value received in step (a) with the processing unit, wherein said processing comprises retrieving from a memory one or more threshold values for the amounts of the biomarkers Osteopontin, cardiac Troponin, a natriuretic peptide and FABP-3 and comparing the value received in step (a) with the one or more threshold values, and
a) providing a prediction of silent infarct and/or cognitive decline via an output device, wherein said prediction is based on the results of step (b).

19. A computer-implemented method of any one of claim 18, wherein said method comprising a further a value for the CHA₂D₂-VASc score is added to receiving at a processing unit in step a).

20. In vitro use of Osteopontin, cardiac Troponin, a natriuretic peptide and FABP-3 or of agents which bind to Osteopontin, cardiac Troponin, a natriuretic peptide and FABP-3 for
a) predicting silent infarcts and/or cognitive decline in a subject,
b) assessing the extent of silent small and large noncortical or cortical infarcts, or
c) improving the prediction accuracy of a clinical stroke risk score for a subject.

21. The method of any one of claims 1 to 20, or the in vitro use of claim 20, wherein
I. Osteopontin is the Osteopontin polypeptide,
II. cardiac Troponin is the cardiac Troponin polypeptide,
III. a natriuretic peptide is the a natriuretic peptide polypeptide,
IV FABP-3 is the FABP-3 polypeptide,
V. the subject is human,
VI. the subject is 65 years or older, and/or
VII. the subject has no known history of stroke and/or TIA (transient ischemic attack)

22. In vitro use of the biomarkers Osteopontin, cardiac Troponin, A natriuretic peptide and FABP-3 or of agents which binds to the biomarker Osteopontin, cardiac Troponin, a natriuretic peptide and FABP-3 for the assessment whether a subject has experienced one or more silent strokes.

23. In vitro use of the biomarkers Osteopontin, cardiac Troponin, A natriuretic peptide and hFABP-3 or of an agent which binds to the biomarkers Osteopontin, cardiac Troponin, A natriuretic peptide and hFABP-3 for improving the prediction accuracy of a clinical stroke risk score for a subject.

All references cited in this specification are herewith incorporated by reference with respect to their entire disclosure content and the disclosure content specifically mentioned in this specification.

### EXAMPLES

### Results

### Example 1: Prediction of Silent Brain Infarcts (LNCCI) Based on Combined Circulating Osteopontin, hsTNT, NT-proBNP and hFABP-3 Levels

The combination of Osteopontin, hsTNT, NT-proBNP and hFABP-3 levels in the assessment of silent brain infarcts provides a method to

1. Predicting the risk of silent brain infarcts in patients with atrial fibrillation based on the combined circulating levels of Osteopontin, hsTNT, NT-proBNP and hFABP-3 in serum/plasma (SWISSAF study, Table 1)

2. Improving the prediction of clinical accuracy of clinical stroke risk scores for silent brain infarcts based on combined circulating levels of Osteopontin, hsTNT, NT-proBNP and hFABP-3 levels in serum/plasma (e.g. CHA₂DS₂-VASc, CHADS2 score) (SWISS AF study, Table 2+3)

The ability of combined circulating levels of Osteopontin, hsTNT, NT-proBNP and hFABP-3 to predict the risk for the occurrence of silent infarcts was assessed in the SWISS AF study (Conen D., Forum Med Suisse 2012, 12:860-862; Conen et al., Swiss Med Wkly. 2017,147). Patients of the SWISS AF cohort have a median age of 74 years, a rate of prior clinical strokes or TIA of 20%, a rate of vascular diseases of 34% and a history of diabetes of 17%. Concentration levels of Osteopontin, hsTNT, NT-proBNP and hFABP-3 were combined using logistic regression model after log-transforming the individual marker concentration levels. Clinical performance for the detection of silent infarcts was asses by ROC analysis and the area under the ROC (AUC)

TABLE 1
Coefficients from the logistic regression model combining the concentration levels
of Osteopontin, hsTNT, NT- proBNP and hFABP-3 into a clinical score. Dependent
variable is the presence of LNCCI lesions.

| | | Standard | | |
|---|---|---|---|---|
| | Coefficient | Error | z-value | P-value |
| (Intercept) | −3.766 | 0.786 | −4.79 | <0.001 |
| hsTNT | 0.374 | 0.153 | 2.452 | 0.014 |
| NT- proBNP | 0.215 | 0.081 | 2.658 | 0.008 |
| hFABP-3 | −0.568 | 0.313 | −1.814 | 0.070 |
| Osteopontin | 0.571 | 0.207 | 2.755 | 0.006 |

Biomarkers and volumes were logarithmized.

Patients with LNCCI on the bMRI were older (75.0 vs 68.1 years, p<0.0001), had more often permanent AF (28.4 vs 17.8%, p=0.0002), higher systolic BP levels (136.7 vs 131.3 mmHg, p<0.0001) and a higher CHA2DS2-VASc score (3.2 vs 2.1 points, p<0.0001), but showed no difference in the rate of oral anticoagulation (90.3 vs 88.5%, p=0.32).

Table 1 shows that Osteopontin, hsTNT, NT-proBNP are positively and significantly associated with the risk of the presence of LNCCI. The coefficient of hFABP-3 is indicating a negative association with the presence of LNCCI and also a p-value slightly above 0.05. However, removing hFABP-3 reduces the clinical performance of the combined model and therefor indication a suppressor effect removing LNCCI unrelated variance from the other biomarkers.

Therefore the risk of silent brain infarcts in patients with atrial fibrillation can be assessed based on the combined circulating levels of Osteopontin, hsTNT, NT-proBNP and hFABP-3 in serum/plasma.

TABLE 2
Coefficients from the logistic regression model combining the concentration levels
of Osteopontin, hsTNT, NT- proBNP, hFABP-3 and CHAD2DS2-VASc into a clinical
score. Dependent variable is the presence of LNCCI lesions.

| | | Standard | | |
|---|---|---|---|---|
| | Coefficient | Error | z-value | P-value |
| (Intercept) | −3.598 | 0.802 | −4.484 | <0.001 |
| CHA2DS2-VASc | 0.083 | 0.07 | 1.183 | 0.237 |
| hsTNT | 0.376 | 0.154 | 2.44 | 0.015 |
| NT- proBNP | 0.194 | 0.083 | 2.343 | 0.019 |
| hFABP-3 | −0.607 | 0.316 | −1.922 | 0.055 |
| Osteopontin | 0.52 | 0.212 | 2.45 | 0.014 |

Biomarkers and volumes were logarithmized.

As shown in Table 2 coefficients for the biomarker stay significant (<0.05)—with the exception of hFABP-3—when a clinical risk score, here in the form CHA2DS2-VASc, is added to the model. On the other hand is visible that the p-value for CHA2DS2-VASc is clearly larger than 0.05 although CHA2DS2-VASc is univariately significantly associated with probability of the presence of LNCCI. This suggests that the biomarker contain more information on the presence of LNCCI than CHA2DS2-VASc.

TABLE 3
Significant improvement of the CHAD2DS2-VASc score with levels of
Osteopontin, hsTNT, NT- proBNP, hFABP-3 for the relation to large
non-cortical infarcts.
Predictor variables were logarithmized biomarkers in addition to
CHADS2-VA2SC score, the outcome variable was presence/absence of
large non-cortical and cortical infarcts.

| Risk Score | AUC |
|---|---|
| CHA2DS2-VASc | 0.602 (0.558; 0.647) |
| Clinical Score (including Osteopontin, | 0.661 (0.616; 0.705) |
| bsTNT, NT- proBNP and hFABP-3) | |
| Clinical Score (including Osteopontin, | 0.665 (0.620; 0.710) |
| hsTNT, NT- proBNP and hFABP-3 ) plus | |
| CHA2DS2-VASc | |

This is also visible when we compare the AUC values of CHA2DS2-VASc (0.602 (0.558; 0.647)) score and the combined model.

The AUC (95% CI) of the CHA2DS2-VASc score was improved by Osteopontin, hsTNT, NT-proBNP, hFABP-3 to 0.665 (0.620; 0.710) as demonstrated in Table 3.

Also the AUC of the model including only the biomarkers Osteopontin, bsTNT, NT-proBNP, hFABP-3 (0.661 (0.616; 0.705)) is superior of CHA2DS2-VASc.

The combination of Osteopontin, hsTNT, NT-proBNP, hFABP-3 with clinical parameters of the CHA2DS2-VASC score well predicted clinically silent brain infarcts and outperformed the CHA2DS2-VASc score. Early clinical identification of patients at risk of cognitive decline might allow for better diagnostic and preventive measures.

These data suggest that the combined circulating levels of Osteopontin, hsTNT, NT-proBNP and hFABP-3 can be used to assess the risk of silent infarcts, to classify the disease, to assess the disease severity, to guide therapy (with objectives to therapy intensification/reduction), to predict disease outcome (risk prediction, e.g. stroke), therapy monitoring (e.g., effect of anti-coagulation drugs on the biomarker clinical risk score levels), therapy stratification (selection of therapy options; e.g. long-term from SWISS AF and selection).

1. A method for assessing whether a subject has experienced one or more silent infarcts and/or for predicting silent infarcts and/or cognitive decline in a subject, said method comprising
a) determining an amount of each of the biomarkers Osteopontin, cardiac Troponin, a natriuretic peptide and FABP-3 in a sample from the subject,
b) comparing the amounts determined in step a) to references, and
c) assessing whether the subject has experienced one or more silent infarcts and/or predicting silent infarcts and/or cognitive decline in the subject.

2. The method of claim 1, wherein amounts of the biomarkers Osteopontin, cardiac Troponin, a natriuretic peptide and FABP-3 larger than the references are indicative of a subject who is suffering from one or more silent infarcts and/or a subject who is likely to suffer from silent infarcts and/or cognitive decline, wherein amounts of the biomarkers Osteopontin, cardiac Troponin, a natriuretic peptide and FABP-3 lower than the references are indicative of a subject who is not suffering from one or more silent infarcts and/or a subject who is not likely to suffer from silent infarcts and/or cognitive decline.

3. (canceled).

4. A method for improving the prediction accuracy of a clinical risk score for silent infarcts and/or cognitive decline for a subject, comprising
a) determining an amount of each of the biomarkers Osteopontin, cardiac Troponin, a natriuretic peptide and FABP-3 in a sample from the subject, and
b) combining a value for the amounts of the biomarkers Osteopontin, cardiac Troponin, a natriuretic peptide and FABP-3 with the clinical risk score for silent infarcts and/or cognitive decline, wherein the prediction accuracy of said clinical risk score for silent infarcts and/or cognitive decline is improved.

5. The method of claim 1, wherein the clinical risk score is a CHA₂DS₂-VASc score, wherein the value for the amounts of the biomarkers Osteopontin, cardiac Troponin, a natriuretic peptide and FABP-3 is combined with the CHA₂DS₂-VASc score, and wherein the prediction accuracy of the clinical risk score for silent infarcts and/or cognitive decline is improved.

6. The method of claim 3, wherein the risk of the subject to suffer from silent infarcts and/or cognitive decline within 1 month to 5 years is predicted.

7. The method of claim 1, wherein the silent infarcts are silent small noncortical and/or cortical infarcts and/or silent large noncortical and/or cortical infarcts.

8. (canceled).

9. The method of claim 1, further comprising
a) recommending anticoagulation therapy,
b) recommending an intensification of anticoagulation therapy,
c) intensified risk factor management, and
d) care in specialized clinics.

10. The method of claim 1, wherein the subject suffers from atrial fibrillation.

11. The method of claim 1, wherein the subject is a human, and/or wherein the sample is selected from blood, serum, plasma, and/or tissue.

12. (canceled).

13. (canceled).

14. (canceled).

15. The method of claim 1, wherein
I. Osteopontin is an Osteopontin polypeptide,
II. cardiac Troponin is a cardiac Troponin polypeptide,
III. a natriuretic peptide is a natriuretic peptide polypeptide,
IV. FABP-3 is a FABP-3 polypeptide,
V. the subject is a human,
VI. the subject is 65 years or older, and/or
VII. the subject has no known history of stroke and/or TIA (transient ischemic attack).

16. The method of claim 1, further comprising combining a value for the amounts of the biomarkers Osteopontin, cardiac Troponin, a natriuretic peptide and FABP-3 with a clinical risk score for silent infarcts, wherein a prediction accuracy of said clinical risk score for silent infarcts is improved

17. The method of claim 16, wherein the clinical risk score is a CHA₂DS₂-VASc score, wherein the value for the amounts of the biomarkers Osteopontin, cardiac Troponin, a natriuretic peptide and FABP-3 is combined with the CHA₂DS₂-VASc score, and wherein a prediction accuracy of the clinical risk score for silent infarcts and/or cognitive decline is improved.

18. The method of claim 1, further comprising monitoring the extent of silent infarcts and/or cognitive decline based on the results of step b).

19. The method of claim 1, wherein determining the amount of Osteopontin in the sample comprises utilizing a sandwich assay comprising a biotinylated first monoclonal antibody that specifically binds Osteopontin and a ruthenylated F(ab′)2-fragment of a second monoclonal antibody that specifically binds Osteopontin, wherein the first antibody and the second antibody form sandwich immunoassay complexes with Osteopontin in the sample,
wherein determining the amount of cardiac Troponin in the sample comprises utilizing a sandwich assay comprising a biotinylated first monoclonal antibody that specifically binds cardiac Troponin and a ruthenylated F(ab′)2-fragment of a second monoclonal antibody that specifically binds cardiac Troponin, wherein the first antibody and the second antibody form sandwich immunoassay complexes with cardiac Troponin in the sample,
wherein determining the amount of a natriuretic peptide in the sample comprises utilizing a sandwich assay comprising a biotinylated first monoclonal antibody that specifically binds the natriuretic peptide and a ruthenylated F(ab′)2-fragment of a second monoclonal antibody that specifically binds the natriuretic peptide, wherein the first antibody and the second antibody form sandwich immunoassay complexes with the natriuretic peptide in the sample, and
wherein determining the amount of FABP-3 in the sample comprises utilizing a sandwich assay comprising a biotinylated first monoclonal antibody that specifically binds FABP-3 and a ruthenylated F(ab′)2-fragment of a second monoclonal antibody that specifically binds FABP-3, wherein the first antibody and the second antibody form sandwich immunoassay complexes with FABP-3 in the sample.

20. The method of claim 4, wherein determining the amount of Osteopontin in the sample comprises utilizing a sandwich assay comprising a biotinylated first monoclonal antibody that specifically binds Osteopontin and a ruthenylated F(ab′)2-fragment of a second monoclonal antibody that specifically binds Osteopontin, wherein the first antibody and the second antibody form sandwich immunoassay complexes with Osteopontin in the sample,
wherein determining the amount of cardiac Troponin in the sample comprises utilizing a sandwich assay comprising a biotinylated first monoclonal antibody that specifically binds cardiac Troponin and a ruthenylated F(ab′)2-fragment of a second monoclonal antibody that specifically binds cardiac Troponin, wherein the first antibody and the second antibody form sandwich immunoassay complexes with cardiac Troponin in the sample,
wherein determining the amount of a natriuretic peptide in the sample comprises utilizing a sandwich assay comprising a biotinylated first monoclonal antibody that specifically binds the natriuretic peptide and a ruthenylated F(ab′)2-fragment of a second monoclonal antibody that specifically binds the natriuretic peptide, wherein the first antibody and the second antibody form sandwich immunoassay complexes with the natriuretic peptide in the sample, and
wherein determining the amount of FABP-3 in the sample comprises utilizing a sandwich assay comprising a biotinylated first monoclonal antibody that specifically binds FABP-3 and a ruthenylated F(ab′)2-fragment of a second monoclonal antibody that specifically binds FABP-3, wherein the first antibody and the second antibody form sandwich immunoassay complexes with FABP-3 in the sample.

21. The method of claim 6, wherein the risk of the subject to suffer from silent infarcts and/or cognitive decline within 1 year or within 2 years is predicted.

22. A method for determining a panel of biomarkers in a subject who has suffered from one or more silent infarcts and/or cognitive decline and/or a subject who is likely to suffer from one or more silent infarcts and/or cognitive decline, the method comprising:
obtaining a sample from the subject;
determining a quantification for a panel of biomarkers in the sample, wherein the panel comprises the biomarkers Osteopontin, cardiac Troponin, a natriuretic peptide, and FABP-3, wherein the quantification comprises determining a level of each of the biomarkers Osteopontin, cardiac Troponin, a natriuretic peptide, and FABP-3 in the panel.

23. The method of claim 22, wherein a level of at least one biomarker in the panel of biomarkers is increased relative to a level of a corresponding biomarker in a reference panel.

24. The method of claim 23, wherein determining the level of at least one biomarker in the panel of biomarkers that is increased relative to the level of the corresponding biomarker in the reference panel is indicative of a subject who has suffered from one or more silent infarcts and/or cognitive decline and/or a subject who is likely to suffer from one or more silent infarcts and/or cognitive decline.

25. The method of claim 22, further comprising combining a value for the levels of the biomarkers Osteopontin, cardiac Troponin, a natriuretic peptide, and FABP-3 with a clinical risk score for silent infarcts and/or cognitive decline, wherein a prediction accuracy of the clinical risk score for silent infarcts and/or cognitive decline is improved.
